(19) United States
(12) Patent Application Publication
(10) Pub. No.: US 2020/0237812 A1
(43) Pub. Date: Jul. 30, 2020

(54) Compound and Composition for Use in the Preventive and/or Curative Treatment of Diseases of the Central Nervous System Characterised by a Decline in Neuronal Plasticity, in Particular Characterised by a Decline in Synaptic Plasticity

(71) Applicant: SYNAPHARM INDUSTRIAL SYNTHESIS, Liège (BE)

(72) Inventors: Philippe DANHIER, Audregnies (BE); Pascale AZZAM, Liège (BE)

(21) Appl. No.: 16/754,994

(22) PCT Filed: Sep. 26, 2018
(86) PCT No.: PCT/EP2018/076133

§ 371 Date: Apr. 9, 2020

(30) Foreign Application Priority Data
Oct. 13, 2017 (BE) 2017/5731

(51) Int. Cl. A61K 33/06; A61K 31/145; A61K 9/48; A61K 45/06; A61P 25/28

(52) U.S. Cl. CPC A61K 33/06; A61K 31/145; A61P 25/28; A61K 45/06; A61K 9/48

(57) ABSTRACT

The present invention concerns a compound and composition for use in the preventive and/or curative treatment of diseases of the central nervous system characterised by a decline in neuronal plasticity, in particular a decline in synaptic plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy, said compound being magnesium N-acetyl taurinate

The present invention concerns a compound and composition for use in the preventive and/or curative treatment of diseases of the central nervous system characterised by a decline in neuronal plasticity, in particular by a decline in synaptic plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy.

“Neuronal plasticity” is a general term which covers the set of physiological, biochemical and anatomical mechanisms that underlie development of the nervous system in the embryo and neonate and which, in adult animals (including human beings), drive mechanisms that lead to regeneration of damaged neurones and restoration of the central nervous system's adaptive capacity when certain parts of it degenerate and cannot regenerate themselves. In consequence, the following processes are involved when it comes to neuronal plasticity: denervation, reinnervation, synaptogenesis, synaptic repression, synaptic expansion, axonal sprouting and neuronal regeneration as well as the development and modelling of neuronal pathways and circuits to replace damaged ones.

It is now accepted that, even in adults, neuronal plasticity is important for the formation of new connections such as those for new neurones (neurogenesis) and glial cells. Nevertheless, ageing and stress factors can compromise this plasticity and lead to the development of cerebral atrophy. Abnormal neuronal plasticity could be one of the factors that contribute to the development of neuropsychiatric disease at different stages of life.

“Synaptic plasticity” is one of the factors (one of ten mechanisms) involved in neuronal plasticity. This means that enhancing/restoring synaptic plasticity helps enhance/restore neuronal plasticity. More specifically, “synaptic plasticity” concerns communication between neurones, in particular the efficiency of synaptic transmission which is directly related to morphological changes in dendritic spines. Dendritic spines are projections from dendrites that form postsynaptic zones which allow the transduction of signals coming from presynaptic neurones. These structures are characterised by their highly plastic nature, their morphology and the fact that their density is modulated by neuronal activity.

It is recognised that “synaptic plasticity” depends on molecular processes that, within the dendritic spines, either maintain optimally efficient synaptic transmission (also referred to as long-term potentiation [LTP]) or reduce the efficiency of synaptic transmission (referred to as long-term depression [LTD]). Signalling pathways that regulate synaptic plasticity involve a great number of receptors, including the ionotropic glutamate NMDAR and AMPAR receptors and the metabotropic mGluR and KA receptors (which are closely related to mGluR).

Long-term modulation of the efficiency of synaptic transmission is one of the basic mechanisms of memorisation and learning. Synchronized activation of pre- and post-synaptic elements enhances the efficiency of synaptic transmission, i.e. induces “long-term potentiation” (LTP). From a cellular or molecular point of view, neuronal plasticity is fast and extensive, with the induction of long-term potentiation being associated with morphological changes in the dendritic spines. It is noted that LTP is defined as a sustained increase in synaptic efficiency following the application of a brief tetanic stimulus to fibers afferent to the recorded structure.

The subjects targeted by the present invention are those suffering from diseases characterised by a decline in neuronal plasticity. Such decline can for example be observed in people who are suffering from degenerative disease of the central nervous system, trauma or cerebral ischaemia. Decline in neuronal plasticity is significantly responsible for memory loss, learning difficulties and the development of certain problems such as autism and dyslexia.

In other words, neuronal plasticity or neuroplasticity designates the brain's ability to remodel its connections, including its synaptic connections, according to the environment and the individual's lived experiences. In the foetus, connections are already being established between neurones. Later, after birth, some of these connections are conserved and others disappear. Neuronal plasticity can come into play in cognitive processes such as learning, perception, attention, reasoning, language and memory, or in disorders or diseases of the central nervous system such as neurodegenerative disease, trauma or cerebral ischaemia, or furthermore in memory problems and attention deficit.

Restoration of neuronal plasticity has been observed in the course of research which showed that training of the target brain can restore cerebral activity and cognitive function in people who have experienced brain injury.

Studies in greater depth of patients' brains showed that, after a suitable brain training programme, previously impaired cognitive performance saw improvement; damaged areas of the brain were reactivated and even, in some cases, regenerated.

This process of neuronal plasticity is mainly made possible by virtue of two changes, namely a morphological change and a change in the electrochemical properties of membranes.

Firstly, the morphological change proceeds through a series of key steps: denervation, reinnervation, synaptogenesis, synaptic repression, synaptic expansion, axonal sprouting and neuronal regeneration coupled with the development and modelling of neuronal pathways and circuits to replace unused and damaged circuits.

Secondly, changes in the membranes' electrochemical properties are observed as changes in action potential along the axon and changes in post-synaptic potentials along dendrites. The propagation of a nervous signal depends on ion channels on the plasma membrane. A change in the density or nature of these ion channels will affect how the electrical signal will propagate. A change in a membrane's electrochemical properties can be short-term if the composition of ion channels is temporarily modified or it can be long-term, usually if one or more types of ion channel are replaced with other types. For example, when new knowledge is acquired, communication or synaptic transmission between the neurones involved is reinforced. Better communication between neurones facilitates the passage of electrical signals along their pathway.

As explained above, there is a real need to stimulate or restore neuronal plasticity in disorders or disease of the central nervous system, such as neurodegenerative disease, trauma or cerebral ischaemia, or also in memory problems and attention deficit. In fact, it has been observed that, in such disorders or diseases, activation and/or restoration of neuronal plasticity in sufferers has a beneficial effect on their state of health by slowing down the decline in brain function.

Non-invasive (non-pharmacological) methods have been developed which have been shown to act by activating and/or enhancing neuronal plasticity. In practice, in order to improve memory or learning processes, it has been shown that the subject has to follow five major rules: (1) avoid stress and get enough sleep, (2) use your brain, (3) eat intelligently, (4) develop a social spirit, and (5) do regular exercise. Although it may be true that a beneficial effect has been observed on neuronal plasticity in people following these five rules, they are often difficult—even impossible—to implement for people suffering from a disorder or disease of the central nervous system. Moreover, in cases like these, the situation is such that it does not simply mean stimulating neuronal plasticity in order to enhance something that is already working fairly well (memory or learning) but rather stimulating—or even restoring—neuronal plasticity in order to create or remodel neurones that have sustained serious damage. In practice, following injury, cerebral ischaemia or neurodegenerative disease, or alternatively in memory problems and attention deficit, neurones are damaged or even completely abolished. Therefore, neuronal plasticity is sought in order to create or repair them.

Pharmacological treatments that promote neuronal plasticity are known, notably from the documents EP0441106 and EP1981522. However, the treatments disclosed in documents EP0441106 and EP1981522 suffer from certain limitations, in particular because of a risk of significant side effects and the perturbation of signalling pathways that are essential for proper functioning of the human body. In addition, such pharmacological treatments are expensive which presents an obstacle for patients and health care insurance systems.

Document US2008/248100 gives a long list of compounds and mentions the use of magnesium taurate (C₄H₁₂MgN₂O₆S₂) for treatment of diseases of the central nervous system characterised by a decline in neuronal plasticity. However, although the treatment proposed by document US2008/248100 minimises the risk of side effects and is supposed to be cheaper, its efficacy remains limited. In practice, it is observed that the bioavailability of magnesium taurate is low as are its bioequivalence and the amount of it that enters cells.

To date, unfortunately, no treatment concerns a compound or composition at an acceptable cost, said compound or composition being effective and safe for the stimulation and/or restoration of neuronal plasticity with satisfactory bioavailability, bioequivalence and intracellular penetration to ensure effective, optimal treatment of diseases characterised by a decline in neuronal plasticity.

There exists therefore a real need to develop and formulate a compound and/or composition to provide an effective, optimal treatment for diseases of the central nervous system characterised by a decline in neuronal plasticity, through activation and/or restoration of neuronal plasticity, said compound and/or composition being of acceptable cost as well as both effective and risk-free (without harmful side effects) for the human body and, above all, presenting satisfactory, optimised bioavailability and/or bioequivalence and/or intracellular penetration.

To solve this problem, the invention provides for a compound and a composition for use in the preventive and/or curative treatment of diseases of the central nervous system characterised by a decline in neuronal plasticity, in particular by a decline in synaptic plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy, said compound being magnesium N-acetyl taurinate. In the framework of the present invention, it was surprisingly ascertained that the compound according to the invention, magnesium N-acetyl taurinate, has superior bioavailability, bioequivalence and intracellular penetration compared to compositions in the background art. Moreover, a composition according to the invention has an acceptable cost and is both effective and safe for the treatment of diseases of the central nervous system characterised by a decline in neuronal plasticity, through the stimulation and/or restoration of neuronal plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy.

A compound according to the invention demonstrates a superior effect on the stimulation and/or restoration of neuronal plasticity for the treatment of a disease or disorder of the central nervous system in a subject by virtue of its dual action. Such a compound according to the invention acts first at a very early stage of neurone repair (genesis of a new neurone) by activating the NMDAR receptor. Secondly, it acts on the production of BDNF (brain-derived neurotrophic factor) that regulates synaptic plasticity in the adult brain.

As mentioned above, it has been demonstrated that a compound according to the invention, namely magnesium N-acetyl taurinate, possesses superior efficacy in terms of bioavailability and bioequivalence. In fact, it is emphasised in the framework of the present invention that the compound according to the invention affords better bioavailability as a result of the action of acetylated taurine on the cell membrane by virtue of its lipophilic nature.

It is noted that magnesium N-acetyl taurinate (C₈H₁₆MgN₂O₈S₂) is known for its protective cytovascular properties such as inhibitory activity on platelet aggregation and protective properties against venous and arterial thrombosis as well as its stabilising activity on the erythrocyte membrane. In addition, magnesium N-acetyl taurinate has been shown to protect against glaucoma by acting on the vascular deregulation as well as it having angioprotective properties through an anti-inflammatory activity based on restoring the levels of Enos (endothelial Nitric Oxide Synths 3). Studies have also shown that magnesium N-acetyl taurinate has a beneficial effect on neurotoxicity due to hyperexcitable ionotropic glutamamatergic receptors. In fact, magnesium N-acetyl taurinate has intraneuronal activity not only on the NMDA receptor but also on two other ionotropic glutamic acid receptors, namely the AMPA and KA receptors that are involved in the speed of synaptic transmission. It has been noticed that such a compound is structurally analogous to both glutamic acid and kainic acid. Thus, magnesium N-acetyl taurinate will target all these glutamatergic receptors, NMDAR, AMPAR and KAR, thereby inhibiting signalling pathways downstream of said receptors.

Highly advantageously, the said magnesium N-acetyl taurinate according to the invention is magnesium N-acetyl taurinate dihydrate which contains two intrinsic water molecules.

The term “two intrinsic water molecules” in the sense of the present invention means that the two water molecules are tightly bound inside the magnesium N-acetyl taurinate dihydrate, i.e. they constitute an integral part of the magnesium N-acetyl taurinate dihydrate molecule as opposed to any water of hydration which could be absorbed or adsorbed by this compound.

The magnesium N-acetyl taurinate dihydrate (C₈H₂₀MgN₂O₁₀S₂), also called Magnesium N-Acetyltaurinate dihydrate, is a magnesium vector and a magnesium analogue of taurine with a molecular weight of 392.677 g/mol, two molecules of water (H₂O) being intrinsic to the magnesium N-acetyl taurinate dihydrate molecule.

In particular, the magnesium N-acetyl taurinate dihydrate is that which is produced and sold under the brand name ATAMg® by the Synapharm Industrial Synthesis company.

Like unhydrated magnesium N-acetyl taurinate, magnesium N-acetyl taurinate dihydrate containing two intrinsic water molecules possesses various characteristics such as a sulphated amine p-derivative, a sulphonic (non-carboxylic) acid, a N-acetylate, and does not present the amphoteric character of taurine (a zwitterion with a positive and a negative charge present on the same group) which optimises intracellular taurinergic activity. Since the electrical charge on the nitrogen of the taurine has been abolished by acetylation, only the electrons of the Mg++ cation are kept chelated by the taurine's two sulphonic groups. This yields an ethanamide (acetamide) derivative which is more lipophilic than amphoteric taurine. This promotes entry across the neuronal phosopholipid membrane. Ethanamide (acetamide) derivatives characterise compounds used for their nootropic (like piracetam), anticonvulsive and anti-epileptic (like levitracitam) activities.

The molecular weight of magnesium N-acetyl taurinate dihydrate is 392.677 g/mol, higher than that of unhydrated magnesium N-acetyl taurinate (C₈H₁₆MgN₂O₈S₂) which is 356.656 g/mol. This form which is not dihydrated is notably described in document FR2384751 in which the magnesium N-acetyl taurinate is obtained by mixing magnesia, taurine, water and acetic acid before two successive drying steps, one in a vacuum at 100° C. and the other with a desiccating solvent in such a way as to generate crystals.

When the compound according to the invention is magnesium N-acetyl taurinate in the dihydrated form containing two intrinsic water molecules, it has been shown that its bioavailability, bioequivalence and ability to enter cells by passing across membranes are further enhanced and optimised by virtue of the presence of the two water molecules intrinsic to the magnesium N-acetyl taurinate dihydrate molecule. The presence of two water molecules helps promote better intracellular penetration of the magnesium N-acetyl taurinate dihydrate.

Moreover, it has been determined that magnesium N-acetyl taurinate in the dihydrated form containing two intrinsic water molecules is more stable over time than non-dihydrated magnesium N-acetyl taurinate, i.e.

than anhydrous magnesium N-acetyl taurinate. In other words, it has been shown that the dihydrated form of magnesium N-acetyl taurinate conserves its properties better over time than the anhydrous form of magnesium N-acetyl taurinate.

It has furthermore been determined that the two intrinsic water molecules are responsible for formation of a stable complex through bonds established between the two intrinsic water molecules and the magnesium ion within the magnesium N-acetyl taurinate dihydrate molecule. More particularly, the magnesium is bound to the N-acetyl taurine and the water through non-covalent bonds, i.e. metal-ligand coordination bonds.

Formation of such a complex is particularly advantageous because it enhances the compound's stability: the magnesium remains anchored to (trapped in) the complex and therefore reaches the target area more efficiently, the target being the brain to drive restoration of neuronal plasticity, in particular restoration of synaptic plasticity, for example in treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy.

The word “bioavailability” in the sense of the present invention describes a pharmacokinetic property of drugs, namely the fraction of a dose that reaches the bloodstream in unchanged form. Therefore, bioavailability gives a measure of the rate of absorption of a drug and the amount absorbed. Thus, the composition according to the invention yields a high percentage of magnesium in the blood which implies more potent magnesium activity on metabolism.

As stated above, it has been shown that the bioavailability and bioequivalence of the magnesium are markedly augmented when the compound according to the invention is magnesium N-acetyl taurinate dihydrate containing two intrinsic water molecules. Intracellular penetration and crossing of the blood-brain barrier are optimised by virtue of the presence of two water molecules intrinsic to the magnesium N-acetyl taurinate dihydrate molecule. The presence of these two water molecules helps promote better intracellular penetration of the magnesium N-acetyl taurinate dihydrate.

It is to be noted that “bioequivalence” reflects metabolic action more accurately and allows the comparison of different products that contain the same active substance (e.g. magnesium).

In addition, as stated above, the compound according to the invention is safe and beneficial (above and beyond its properties detailed above) for the human body. In practice, magnesium contributes to maintaining fluid and electrolyte balance, to normal energy metabolism and protein synthesis, to adapted muscle function, to reducing tiredness and asthenia, to maintaining healthy teeth and good bone structure, and to normal functioning of the nervous system and psychological function.

Moreover, the N-acetyl taurinate promotes the intracellular penetration of magnesium and taurine as well as acting on the maintenance of cellular osmolarity.

Preferably, the compound is presented in a form that can be administered orally, for example in the form of a tablet, a capsule, a gelcap, a soluble powder, an oily solution, an effervescent tablet or a soft capsule.

In a particular form according to the invention, the compound is administered as 1-2 capsules a day in adults, teenagers and children.

Other embodiments of the compound according to the invention are indicated in the appended Claims.

The present invention also concerns a composition for use in the preventive and/or curative treatment of diseases of the central nervous system characterised by a decline in neuronal plasticity, in particular by a decline in synaptic plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy, said composition containing at least some magnesium N-acetyl taurinate.

Highly advantageously, in a composition according to the invention, said magnesium N-acetyl taurinate is magnesium N-acetyl taurinate dihydrate containing two intrinsic water molecules.

The term “two intrinsic water molecules” in the sense of the present invention means that the two water molecules are tightly bound inside the magnesium N-acetyl taurinate dihydrate molecule, i.e. they constitute an integral part of the magnesium N-acetyl taurinate dihydrate as opposed to any water of hydration which could be absorbed or adsorbed by this compound.

The magnesium N-acetyl taurinate dihydrate (C₈H₂₀MgN₂O₁₀S₂), also called Magnesium N-Acetyltaurinate dihydrate, is a magnesium vector and a magnesium analogue of taurine with a molecular weight of 392.677 g/mol, two molecules of water (H₂O) being intrinsic to the magnesium N-acetyl taurinate dihydrate molecule.

In particular, the magnesium N-acetyl taurinate dihydrate is that which is produced and sold under the brand name ATAMg® by the Synapharm Industrial Synthesis company.

The molecular weight of magnesium N-acetyl taurinate dihydrate is 392.677 g/mol, higher than that of unhydrated magnesium N-acetyl taurinate (C₈H₁₆MgN₂O₈S₂) which is 356.656 g/mol. This form which is not dihydrated is notably described in document FR2384751 in which the magnesium N-acetyl taurinate is obtained by mixing magnesia, taurine, water and acetic acid before two successive drying steps, one in a vacuum at 100° C. and the other with a desiccating solvent in such a way as to generate crystals.

When, in a composition according to the invention, the magnesium N-acetyl taurinate is magnesium N-acetyl taurinate dihydrate containing two intrinsic water molecules, it has been shown that its bioavailability, its bioequivalence and its intracellular penetration by crossing membranes are further enhanced and optimised by virtue of the presence of two water molecules intrinsic to the magnesium N-acetyl taurinate dihydrate molecule. The presence of these two water molecules helps promote better intracellular penetration of the magnesium N-acetyl taurinate dihydrate.

It has also been determined that the two intrinsic water molecules are responsible for forming a stable complex by making bonds between the two intrinsic water molecules and the magnesium ion within the magnesium N-acetyl taurinate dihydrate molecule. More particularly, the magnesium is bound to the N-acetyl taurine and the water through non-covalent bonds, i.e. metal-ligand coordination bonds.

The formation of such a complex is particularly advantageous since it confers enhanced stability on the compound: the magnesium remains anchored to (trapped inside) the complex and thereby reaches the target area all the more efficiently, the target being the brain in order to induce restoration of neuronal plasticity neuronal plasticity, in particular the restoration of synaptic plasticity, for example in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy.

Advantageously, the magnesium N-acetyl taurinate or the magnesium N-acetyl taurinate dihydrate containing two intrinsic water molecules is present at a concentration of 40-60% by weight with respect to the overall weight of the composition.

In a particularly advantageous form, the composition according to the invention also contains taurine at a concentration of 10-15% by weight with respect to the overall weight of the composition.

Advantageously, the composition according to the invention also contains an additional magnesium compound selected from the group of magnesium salts of amino acids, magnesium citrate, magnesium gluconate, magnesium lactate, magnesium malate, magnesium taurate, magnesium bisglycinate, magnesium glycerophosphate and magnesium threonate as well as derivatives and mixtures thereof.

Preferably, according to the invention, the composition also contains magnesium at a concentration of 8-15% by weight with respect to the overall weight of the composition.

Moreover, in a particular embodiment, the composition also contains at least one amino acid, e.g. threonine.

Preferably, said at least one amino acid is glycine present at a concentration of 10-15% by weight with respect to the overall weight of the composition.

Advantageously, said at least one amino acid is threonine present at a concentration of 10-15% by weight with respect to the overall weight of the composition.

In a particular form according to the invention, the composition also contains at least one vitamin selected from the group consisting of Vitamin B1, B2, B6 and mixtures thereof.

Advantageously, according to the invention, said at least one vitamin is Vitamin B1 present at a concentration of 0.05-0.1% by weight with respect to the overall weight of the composition.

Preferably, said at least one vitamin is Vitamin B2 present at a concentration of 0.05-0.1% by weight with respect to the overall weight of the composition.

In a particularly advantageous embodiment according to the invention, said at least one vitamin is Vitamin B6 present at a concentration of 0.05-0.15% by weight with respect to the overall weight of the composition.

Advantageously, the composition also contains at least one pharmaceutically acceptable excipient, preferably four pharmaceutically acceptable excipients selected from the group consisting of glyceryl behenate, sucrose stearate, microcrystalline cellulose, magnesium stearate, silicon dioxide and pea maltodextrin and mixtures thereof.

In a particular form, the composition according to the invention is presented in the form of a medicinal product.

Preferably, the composition according to the invention is presented in the form of a dietary supplement.

Advantageously, the composition according to the invention is presented in the form of a drink.

In a particularly advantageous embodiment according to the invention, the composition is presented in a form for oral administration, for example as a tablet, capsule, gelcap, pill, soluble powder, oily solution, effervescent tablet or soft capsule.

Moreover, in a particular embodiment, the composition according to the invention is administered as 1-2 capsules a day to adults, teenagers and children.

Other embodiments of the composition according to the invention are presented in the appended claims.

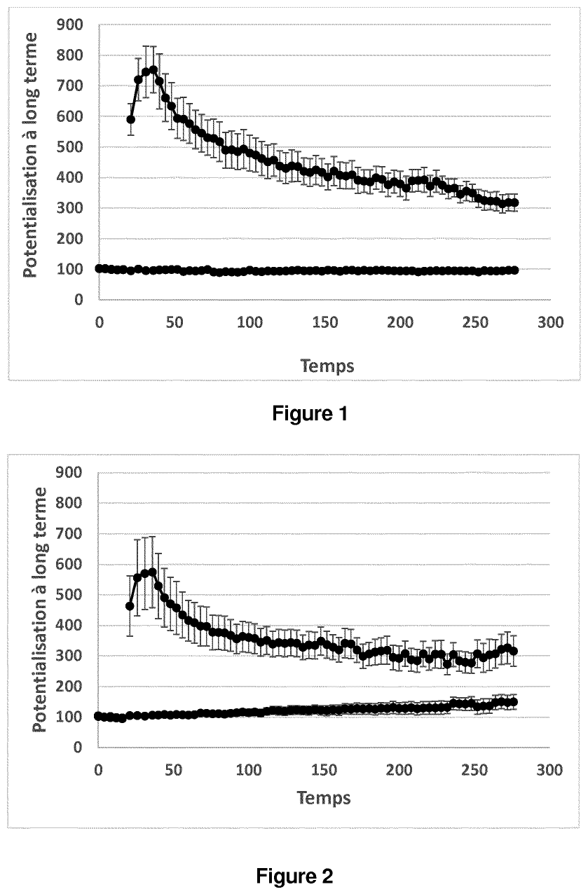

FIG. 1 shows long-term potentiation (%) as measured in rats being fed a normal diet.

FIG. 2 shows long-term potentiation (%) in magnesium-deficient rats being fed a low-magnesium diet.

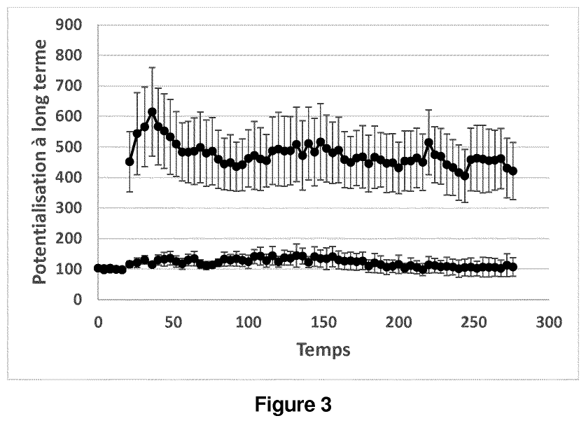

FIG. 3 shows long-term potentiation (%) in magnesium-deficient rats which have been supplemented with a composition according to the invention consisting of magnesium N-acetyl taurinate dihydrate containing two intrinsic water molecules (ATAMg dihydrate) at a dosage of 50 mg/kg/day magnesium N-acetyl taurinate dihydrate containing two intrinsic water molecules.

### EXAMPLE

### 1. Effect of a Composition According to the Invention Consisting of Magnesium N-Acetyl Taurinate Dihydrate Containing Two Intrinsic Water Molecules on the Restoration of Synaptic Plasticity

Comparative experiments were conducted on rats to measure the efficiency of synaptic transmission and synaptic plasticity, in particular in order to measure the recovery of synaptic plasticity.

Three groups of rats were made up as follows:
Group 1: control rats on a normal diet;
Group 2: magnesium-deficient rats on a low-magnesium diet;
Group 3: magnesium-deficient rats supplemented with a composition according to the invention containing magnesium N-acetyl taurinate dihydrate containing two intrinsic water molecules (ATAMg dihydrate) at a dose of 50 mg/kg/hour magnesium N-acetyl taurinate dihydrate containing two intrinsic water molecules (in the water). Supplementation was started 40 days after the beginning of the low-magnesium diet and was carried on for 24 days.

Table 1 below shows the amounts of magnesium (mg/kg/jour) ingested by the rats in each Group in the water and feed given to them.

TABLE 1

| | | | ATAMg | Total |
|---|---|---|---|---|
| | | | dihydrate | amount |
| | Feed | Water | in the water | of Mg |
| Group 1 | 21 | 1.15 | 0 | 22.15 |
| Group 2 | 1.5 | 1.15 | 0 | 2.65 |
| Group 3 | 1.5 | 1.15 | 3.7 | 6.35 |

The hippocampi of the rats from each group were dissected to measure synaptic plasticity. In particular, synaptic responses were measured between Schaffer collaterals and dendrites in the CA1 region. A baseline was recorded for 30 minutes corresponding to a synaptic response of 40% of the peak response. Then the long-term plasticity process was induced by applying four 100 Hz trains at 5-minute intervals. The increased response was measured over four hours. For every experiment, a control pathway without any high-frequency stimulation was also measured.

FIGS. 1-3 show results obtained in each group. These figures show that:
for rats in Group 1 (control group), a very stable control pathway is observed as well as long-term potentiation of over 300% (FIG. 1);
for rats in Group 2 (magnesium-deficient rats), an unstable control pathway is observed and this rises with time; the long-term potentiation observed is below 300% (FIG. 2);
for rats in Group 3 (magnesium-deficient rats supplemented with a composition according to the invention containing magnesium N-acetyl taurinate dihydrate with two intrinsic water molecules), stabilisation of the baseline is observed as well as long-term potentiation of over 300% (FIG. 3).

These findings—especially comparison of the results from Groups 2 and 3—point up the fact that a composition according to the invention containing magnesium N-acetyl taurinate dihydrate with two intrinsic water molecules induces the recovery/restoration of synaptic plasticity.

The present invention has been described in terms of specific embodiments which have purely illustrative value and should not be considered as exhaustive. In general, it would seem evident to those skilled in the art that the present invention is not limited to the examples illustrated and/or described above.

Use of the terms “containing”, “including”, “consisting of”, “with” and any other variant or conjugation thereof cannot in any way exclude the presence of elements other than those mentioned.

Use of the indefinite articles “a” or “an”, or the definite article “the”, to qualify an element does not rule out the possibility that said elements may be plural.

1. Compound for use in the preventive and/or curative treatment of diseases of the central nervous system characterised by a decline in neuronal plasticity, in particular characterised by a decline in synaptic plasticity, including in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy, said compound being magnesium N-acetyl taurinate.

2. Compound for use according to claim 1, said magnesium N-acetyl taurinate being magnesium N-acetyl taurinate dihydrate containing two intrinsic water molecules.

3. Compound for use according to claim 1, said compound being presented in a form that can be administered orally, including in the form of a tablet, a capsule, a gelcap, a soluble powder, an oily solution, an effervescent tablet or a soft capsule.

4. Compound for use according to claim 1, said compound to be administered as 1-2 capsules a day in adults, teenagers and children.

5. Composition for use in the preventive and/or curative treatment of diseases of the central nervous system characterised by a decline in neuronal plasticity, in particular characterised by a decline in synaptic plasticity, including in the treatment of diseases of the central nervous system affecting cognitive processes such as learning, perception, attention, reasoning, language and/or memory, in the treatment of diseases of the central nervous system such as neurodegenerative diseases including Parkinson's disease, Alzheimer's disease and/or amyotrophic lateral sclerosis, in the treatment of diseases of the central nervous system responsible for mood disorders, hyperexcitability, hyperactivity and/or stress, and in the treatment of neurological conditions such as epilepsy, said composition containing at least some magnesium N-acetyl taurinate.

6. Composition for use according to claim 5, characterised in that the said magnesium N-acetyl taurinate is magnesium N-acetyl taurinate dihydrate containing two intrinsic water molecules.

7. Composition for use according to claim 5, in which said magnesium N-acetyl taurinate or magnesium N-acetyl taurinate dihydrate containing two intrinsic water molecules is present at a concentration of 40-60% by weight with respect to the overall weight of the composition.

8. Composition for use according to claim 5, which contains, among other ingredients, taurine at a concentration of 10-15% by weight with respect to the overall weight of the composition.

9. Composition for use according to claim 5, which contains, among other ingredients, an additional magnesium compound selected from the group of magnesium salts of amino acids, magnesium citrate, magnesium gluconate, magnesium lactate, magnesium malate, magnesium taurate, magnesium bisglycinate, magnesium glycerophosphate and magnesium threonate as well as derivatives and mixtures thereof.

10. Composition for use according to claim 5, which contains, among other ingredients, magnesium at a concentration of 8-15% by weight with respect to the overall weight of the composition.

11. Composition for use according to claim 5, which contains, among other ingredients, at least one amino acid, for example threonine.

12. Composition for use according to claim 11 in which said at least one amino acid is present at a concentration of 10-15% by weight with respect to the overall weight of the composition.

13. Composition for use according to claim 5, which contains, among other ingredients, at least one vitamin selected from the group consisting of Vitamin B1, B2, B6 and mixtures thereof.

14. Composition for use according to claim 13 in which said at least one vitamin is present at a concentration of 0.05-0.15% by weight with respect to the overall weight of the composition.

15. Composition for use according to claim 5, containing, among other things, at least one pharmaceutically acceptable excipient selected from the group consisting of glyceryl behenate, sucrose stearate, microcrystalline cellulose, magnesium stearate, silicon dioxide and pea maltodextrin, and mixtures thereof.

16. Composition for use according to claim 5, said composition being presented in the form of a medicinal product, a dietary supplement or a drink.

17. Compound for use according to claim 5, said composition being presented in a form for oral administration, for example as a tablet, capsule, gelcap, pill, soluble powder, oily solution, effervescent tablet or soft capsule.

18. Composition for use according to claim 5, said composition to be administered as 1-2 capsules a day to adults, teenagers and children.

19. Composition for use according to claim 15, containing four pharmaceutically acceptable excipients.
